(19) United States
(12) Patent Application Publication
(10) Pub. No.: US 2009/0048657 A1
(43) Pub. Date: Feb. 19, 2009

(54) Preferentially varying-density ePTFE structure

(75) Inventors: Julio Duran, Morris Plains, NJ (US); Jamie S. Henderson, Oakland, NJ (US); Krzysztof Sowinski, Lodz (PL)

Correspondence Address: HOFFMANN & BARON, LLP, 6900 JERICHO TURNPIKE, SYOSSET, NY, 11791, US

(73) Assignee: Boston Scientific Scimed, Inc.

(21) Appl. No.: 11/893,141

(22) Filed: Aug. 15, 2007

(51) Int. Cl. A61F 2/06

(52) U.S. Cl. 623/1.13

(57) ABSTRACT

The ePTFE structure has a node and fibril micro-structure. The ePTFE structure includes first and second regions each of which has a corresponding density. The density of the first region is different from the density of the second region.

### FIELD OF THE INVENTION

The present invention relates generally to structures containing expanded polytetrafluoroethylene (ePTFE) and methods for making the same. More specifically, the present invention relates to ePTFE structures having regions of different densities, and methods for making such ePTFE structures.

### BACKGROUND OF THE INVENTION

It is known to use extruded tube structures of ePTFE as implantable intraluminal prostheses, particularly as grafts for vascular, esophageal, ureteral and enteral applications. ePTFE is particularly suitable as an implantable prosthesis as it exhibits superior biocompatibility. ePTFE tube structures may be used as vascular grafts in the replacement or repair of a blood vessel as ePTFE exhibits low thrombogenicity. In vascular applications, the grafts are manufactured from ePTFE tube structures which have a microporous micro-structure. This micro-structure allows natural tissue ingrowth and cell endothelization once implanted in the vascular system. This contributes to long term healing and patency of the graft. Vascular grafts formed of ePTFE have a porous fibrous state which is defined by the interspaced nodes interconnected by elongated fibrils.

Grafts formed of ePTFE have a fibrous state which is defined by interspaced nodes interconnected by elongated fibrils. The fibril state of the ePTFE includes interior pores or voids which provides the ePTFE with porosity. Porosity typically enhances tissue ingrowth and cells endothelization.

Microporous ePTFE tubes for use as vascular grafts are known. The porosity of an ePTFE vascular graft may be controllably varied by controllably varying the density. For example, a decrease in the density within a given structure may result in an increased porosity, i.e., increased pore size, which, in turn, results in larger voids in the ePTFE material. Increased porosity typically enhances tissue ingrowth as well as cell endothelization along the inner and outer surface of the ePTFE tube.

Decreasing the density of an ePTFE tube, however, may limit other properties of the tube. For example, decreasing the density of the tube may reduce the overall radial and tensile strength thereof as well as reduce the ability of the graft to retain a suture placed in the tube during implantation. Such a suture typically extends through the wall of the graft. Also, such microporous tubes tend to exhibit low axial tear strength, so that a small tear or nick will tend to propagate along the length of the tube. Thus, if the ePTFE tube has a uniform density along its length, the minimum density thereof may be limited by the strength requirements of the tube.

### SUMMARY OF THE INVENTION

The ePTFE structure of the present invention has a node and fibril microstructure. The ePTFE structure includes first and second regions each of which has a corresponding density. The density of the first region is different from the density of the second region.

The difference in the densities of the first and second regions results in differences in the characteristics and properties thereof. Such a characteristic or property which differs between the first and second regions may be the respective porosities thereof.

The difference in the densities of the first and second regions enables the formation of a vascular graft selected regions of which have respective properties, the combination of which may be difficult to provide in a single graft made according to conventional techniques. Thus, for example, a single graft of the present invention may have some regions with a high porosity and other regions with a low porosity.

The regions of the vascular graft which have a low density provides sites for enhanced tissue ingrowth as well as cell endothelization. This increases the stability of the graft within the human body. Also, the less dense region is more porous and will allow the passage of liquids such as blood or drugs. The high porosity of the reduced density region of the ePTFE has an increased level and rate of ingrowth of tissue over time. This benefits the vascular graft by acting as an anchoring point for the graft as well as a stent which may be secured thereto. This benefit is especially advantageous to an ePTFE tubular structure which covers a stent where the potential of migration is preferably limited.

The regions of the vascular graft which have a low density have increased flexibility. Increased flexibility provides resistance to kinking of the vascular graft.

The characteristics and properties of the regions of the ePTFE structure which have an increased density include increased strength. The increased strength of the ePTFE structure typically provides resistance to propagation of a tear through the ePTFE structure which may result from the piercing of the structure associated with the insertion of a suture through the ePTFE structure. Insertion of a suture through the ePTFE structure may be included in a method for implanting the ePTFE structure in the tissue of a patient. If the region of the graft to be pierced can be identified just prior to the piercing, then other longitudinal regions of the graft may have lower strength requirements and therefore have a reduced density. A further benefit of increased strength of the ePTFE structure is increased durability thereof.

The different characteristics and properties resulting from the difference in densities of the first and second regions provides for the vascular graft to have specific longitudinal regions having increased densities and associated strengths. The same vascular graft can have other specific longitudinal regions which have a low density, even if the other specific longitudinal regions have limited strength. The strength may be provided to the vascular graft by the specific longitudinal regions having increased densities where such specific longitudinal regions have an annular cross-section and accordingly, the shape of individual rings. Such longitudinal regions may typically be spaced apart from one another longitudinally and nevertheless provide the necessary strength to the vascular graft. Therefore, the regions of the graft between the strengthened axial regions may have a lower requirement for strength and may therefore have a reduced density. A low density provides sites for enhanced tissue ingrowth as well as cell endothelization.

The present invention includes methods for making the ePTFE structure which has regions of different densities. One such method includes heating the ePTFE structure to provide the regions which have different densities. Other methods include making intermediates which facilitate the subsequent manufacture of the ePTFE structure which has regions of different densities. One such method includes extruding a PTFE billet to form a PTFE structure having regions of different densities. Another such method includes compacting a PTFE resin to form a PTFE billet which has regions of different densities. These methods enable the formation of an ePTFE structure, selected regions of which have respective densities, the combination of which may be difficult to provide in a single ePTFE structure made according to conventional processes.

These and other features of the invention will be more fully understood from the following description of specific embodiments of the invention taken together with the accompanying drawings.

### BRIEF DESCRIPTION OF THE DRAWINGS

In the drawings:

FIG. 1 is a side elevational view of the preferentially varying-density ePTFE structure of the present invention, the ePTFE structure being shown as tubular and including longitudinal regions which have different densities;

FIG. 2 is a transverse cross-sectional view of the ePTFE tubular structure of FIG. 1 in the plane indicated by line 2-2 of FIG. 1;

FIG. 3 is an enlarged schematic view of the ePTFE tubular structure of FIG. 1, the ePTFE tubular structure being shown as having regions of different densities related to the distances between the nodes of the node and fibril microstructure;

FIG. 4 is a photomicrograph of a region of the ePTFE tubular structure of FIG. 1 which has a relatively high density;

FIG. 5 is a photomicrograph of a region of the ePTFE tubular structure of FIG. 1 which has a relatively low density;

FIG. 6 is an enlarged schematic view of an alternative second embodiment of the ePTFE tubular structure of FIG. 1, the ePTFE tubular structure being shown as having regions of different densities related to the sizes of the nodes of the node and fibril microstructure;

FIG. 7 is an enlarged schematic view of an alternative third embodiment of the ePTFE tubular structure of FIG. 1, the ePTFE tubular structure being shown as having regions of different densities related to the lengths of the fibrils of the node and fibril microstructure;

FIG. 8 is an enlarged schematic view of an alternative fourth embodiment of the ePTFE tubular structure of FIG. 1, the ePTFE tubular structure being shown as having regions of different densities related to the sizes of the resin particles thereof;

FIG. 9 is an enlarged schematic view of an alternative fifth embodiment of the ePTFE tubular structure of FIG. 1, the ePTFE tubular structure being shown as having regions of different densities related to the orientation of the nodes of the node and fibril microstructure;

FIG. 10 is a side elevational view of a stent-graft composite including an alternative sixth embodiment of the ePTFE tubular structure of FIG. 1 assembled to a stent structure, the outer surface of the ePTFE tubular structure being shown as having regions of different densities;

FIG. 11A is an enlarged schematic view of a section of the outer surface of the ePTFE tubular structure of FIG. 10 showing the regions of different densities;

FIG. 11B is an enlarged schematic view of a section of the outer surface of the ePTFE tubular structure of FIG. 10 showing the regions of different densities by shading;

FIG. 12 is an enlarged schematic view of an alternative seventh embodiment of the ePTFE tubular structure of FIG. 1, the ePTFE tubular structure being shown as including radial regions which have different densities;

FIG. 13 is a transverse cross-sectional enlarged schematic view of the ePTFE tubular structure of FIG. 12 in the plane indicated by line 13-13 of FIG. 12, the ePTFE tubular structure being shown as having regions of different densities related to the distances between the nodes of the node and fibril microstructure;

FIG. 14 is a block diagram showing a method for making a preferentially varying-density ePTFE tubular structure of the present invention, the method being shown as including differentially heating respective regions of the ePTFE tubular structure;

FIG. 15 is a block diagram showing an alternative second embodiment of the method of FIG. 14, the method being shown as including differentially extruding respective regions of a PTFE billet; and

FIG. 16 is a block diagram showing an alternative third embodiment of the method of FIG. 14, the method being shown as including differentially compacting a PTFE resin.

Corresponding reference characters indicate corresponding parts throughout the several views of the drawings.

### DETAILED DESCRIPTION OF THE INVENTION

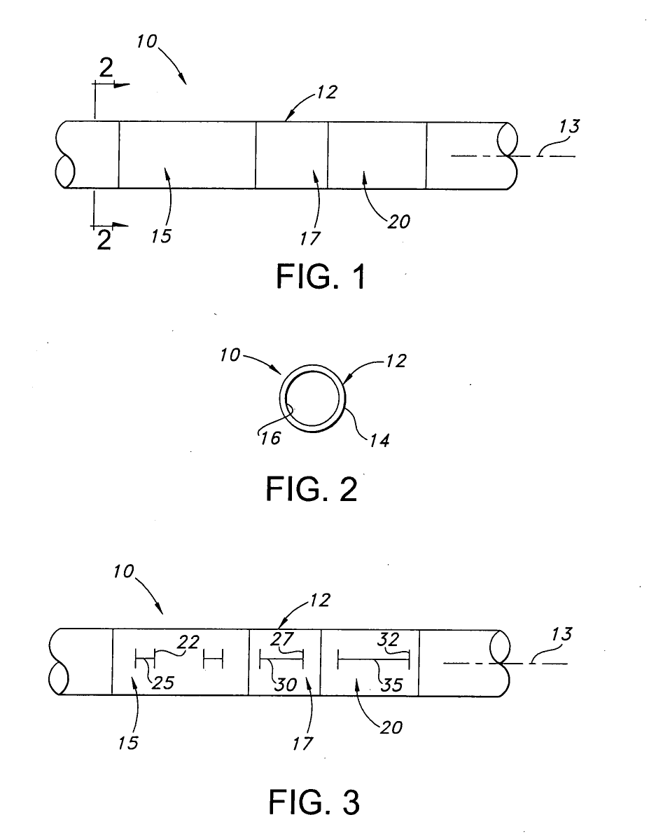

Referring to the drawings and more particularly to FIGS. 1 and 2, a preferentially varying-density ePTFE structure 10 is shown for implantation within a body. The ePTFE structure 10 shown in FIG. 1 is a tubular structure 12 which has a longitudinal axis 13, and outer and inner surfaces 14, 16. The tubular structure 12 has an annular or ring shaped cross-section, the outer and inner diameters of which are substantially constant along the length of the tubular structure. In alternative embodiments, the ePTFE structure 10 may have non-tubular structures such as a plate or a fiber which has a solid or continuous or closed cross-section. The ePTFE structure 10 is formed of homogeneous material having a fibrous state which is defined by interspaced nodes which are interconnected by elongated fibrils, referred to herein as a “node and fibril microstructure”.

The tubular structure 12 has longitudinal first, second, and third regions 15, 17, 20. The first, second, and third regions 15, 17, 20 have corresponding first, second, and third longitudinal positions relative to the longitudinal axis 13. The first, second, and third longitudinal positions of the first, second and third regions 15, 17, 20 are different from one another. The first, second, and third regions 15, 17, 20 each have annular cross-sections such that each of the regions has a ring shape, as shown in FIG. 2. The respective outer and inner diameters of each of the regions 15, 17, 20 are the same. The respective lengths of the regions 15, 17, 20 are different.

The first, second, and third regions 15, 17, 20 each has a corresponding density. The respective densities of the first, second, and third regions 15, 17, 20 are different from one another. The differences in the densities of the first, second, and third regions 15, 17, 20 are provided by controlling specific characteristics or aspects of the node and fibril microstructure of the tubular structure 12. Once such characteristic of the node and fibril microstructure of the tubular structure 12 which may be controlled to vary the density of the first, second, and third regions 15, 17, 20 is the internodal distance (IND) which is the distance between adjacent nodes. Variation in the IND among the first, second, and third regions 15, 17, 20 is illustrated schematically in FIG. 3. In FIG. 3, the first region 15 is shown to have nodes 22 which are separated by a first distance or IND 25. The second region 17 is shown as having nodes 27 which are separated by a second distance or IND 30. The third region 20 is shown as having nodes 32 which are separated by a third distance or IND 35. The first, second, and third distances 25, 30, 35 are related to the respective densities of the first, second and third regions 15, 17, 20. The first, second, and third distances 25, 30, 35 are different from one another which provides for the densities of the first, second, and third regions 15, 17, 20 to be different from one another.

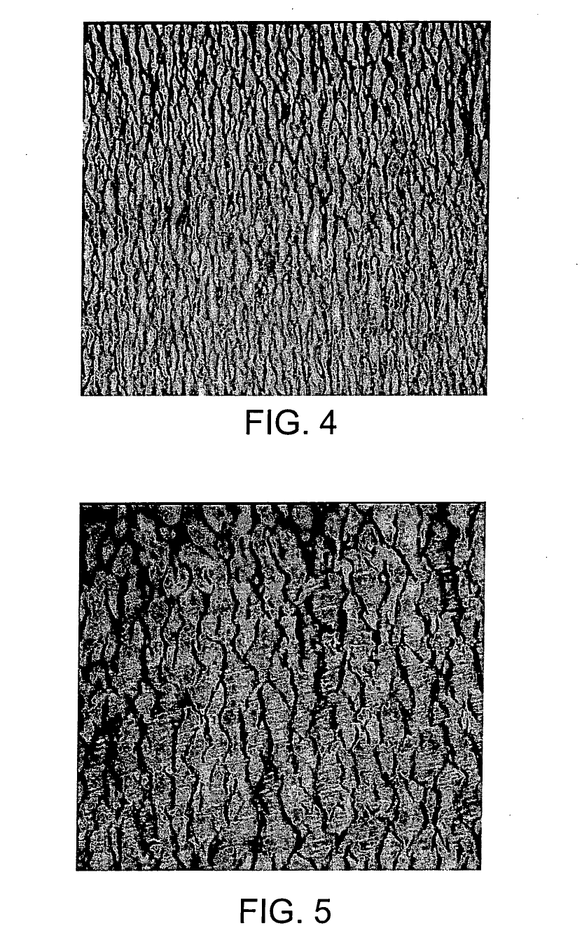

FIGS. 4 and 5 are photomicrographs of node in fibril microstructures of ePTFE which show differences in the distances between the nodes. More specifically, the distances between the nodes shown in FIG. 4 are less than the distances between the nodes shown in FIG. 5. Consequently, the nodes shown in FIG. 5 are less compact than the nodes shown in FIG. 4. As a result, the node in fibril microstructure shown in FIG. 4 has a higher density relative to the node in fibril microstructure shown in FIG. 5. Also, the node in fibril microstructure shown in FIG. 5 has a higher porosity relative to the microstructure shown in FIG. 4.

The relative distances between the nodes and densities of the node and fibril microstructures shown in FIGS. 4 and 5 are illustrative of the differences between the node and fibril microstructures of the first, second, and third regions 15, 17, 20. For example, the first region 15 could be as shown in FIG. 4 and the node and fibril microstructures of either the second or third regions 17, 20 could be as illustrated in FIG. 5 since the distance 25 between the nodes 22 is less than both of the distances 30, 35 between the nodes 27, 32, respectively. Also, the density of the first region 15 is greater than both of the densities of the second and third regions 17, 20, respectively.

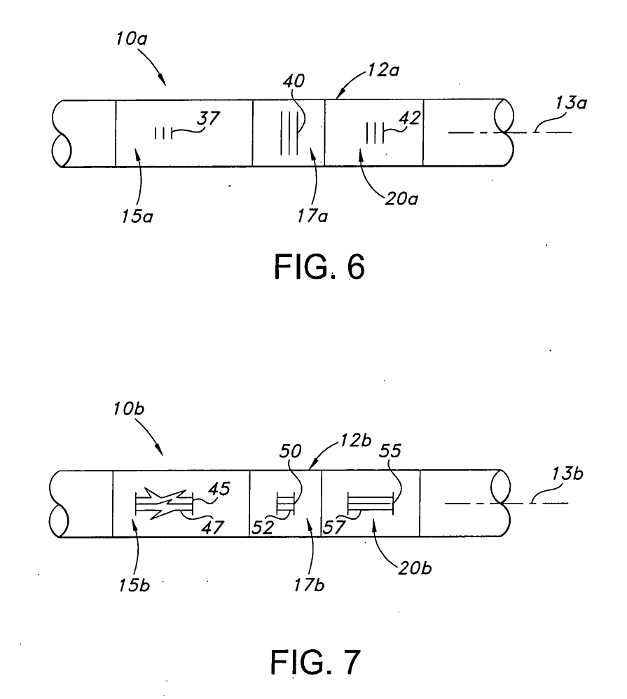

An alternative embodiment of the ePTFE structure 10a is shown in FIG. 6. Parts illustrated in FIG. 6 which correspond to parts illustrated in FIGS. 1 to 3 have, in FIG. 6, the same reference numeral as in FIGS. 1 to 3 with the addition of the suffix “a”. The node and fibril microstructure of the first region 15a includes a plurality of nodes 37 each of which has a first size. The node and fibril microstructure of the second region 17a includes a plurality of nodes 40 each of which has a second size. The node and fibril microstructure of the third region 20a includes a plurality of nodes 42 each of which has a third size. The first, second, and third sizes of the respective nodes 37, 40, 42 are related to the respective densities of the first, second, and third regions 15a, 17a, 20a. The first, second, and third sizes of the nodes 37, 40, 42 are different from one another which provides for the densities of the first, second, and third regions 15a, 17a, 20a to be different from one another.

An alternative embodiment of the ePTFE structure 10b is shown in FIG. 7. Parts illustrated in FIG. 7 which correspond to parts illustrated in FIGS. 1 to 3 have, in FIG. 7, the same reference numeral as in FIGS. 1 to 3 with the addition of a suffix “b”. The node and fibril microstructure of the first region 15b includes a plurality of nodes 45 connected to one another by a plurality of fibrils 47, each of which has a first length. The fibrils 47 have non-linear configurations, such as by being bent or jagged. Such non-linear configurations of the fibrils 47 may be provided by the node and fibril microstructure of the first region 15b being subjected to a compression or shrinkage process. Such a process results in the distance between the nodes 45 being reduced which, in turn, typically forces the fibrils 47 to assume non-linear configurations, such as shown in FIG. 7. These non-linear configurations of the fibrils 47 result from the length thereof not typically being able to be reduced appreciably. Consequently, a reduction in the distance between the nodes 45 results in the fibrils 47 being bent or folded, as shown in FIG. 7.

The second region 17b includes a plurality of nodes 50 connected to one another by a plurality of fibrils 52 each of which has a second length. The fibrils 52 have a linear configuration. The second length of the fibrils 52 is less than the first length of the fibrils 47 as a result of the distance between the nodes 50 being less than the distance between the nodes 45 and the linear configuration of fibrils 52 as compared to the non-linear configuration of the fibrils 47. The lengths and configurations of the fibrils 47, 52 are related to the number of voids in the first and second regions 15b, 17b. Typically, fibrils which have a relatively long length are connected to nodes which are separated by larger distances. This results in the formation of more voids during the initial expansion and microstructure development of the ePTFE structure 10b. An increased number of voids provides for more air within the microstructure which results in a reduced density of the ePTFE structure 10b. Conversely, fibrils which have a relatively short length are typically connected to nodes which are separated by smaller distances resulting in the formation of fewer voids which provide an increased density of the ePTFE structure 10b. This typical relation between the fibril length and density may be complicated by the non-linear configuration of the fibrils 47.

The third region 20b includes a plurality of nodes 55 connected to one another by a plurality of fibrils 57 each of which has a third length. The fibrils 57 have a linear configuration. The third length of the fibrils 57 is greater than the second length of the fibrils 52 as a result of the distance between the nodes 55 being greater than the distance between the nodes 50, and the fibrils 52, 57 both having linear configurations. The larger length of the fibrils 57 relative to the length of the fibrils 52 provides for the density of the second region 17b to be greater than the density of the third region 20b.

In an alternative embodiment, it is possible for the length of the fibrils 57 to be less than the length of the fibrils 52 if the fibrils 52 have non-linear configurations, such as the configurations of the fibrils 47. In such an alternative embodiment, the greater length of the fibrils 52 relative to the length of the fibrils 57 may result from the fibrils 52 being extended to a length which is greater than the length of the fibrils 57 and the second region 17b being subsequently subjected to a compression or shrinkage process which is sufficient to provide the distance between the nodes 50 shown in FIG. 7.

The length of the fibrils 57 is less than the length of the fibrils 47. This illustrates how fibril length can differ in microstructures in which the respective INDs are the same since the distance between the nodes 45 is the same as the distance between the nodes 55. The non-linear configuration of the fibrils 47 may have resulted from extension of the fibrils 47 to a linear configuration in which the length of the fibrils 47 was greater than the length of the fibrils 57. Subsequently, the microstructure of the first region 15b may have been subjected to a compression or shrinkage process which was sufficient to provide the distance between the nodes 45 to be the same as the distance between the nodes 55.

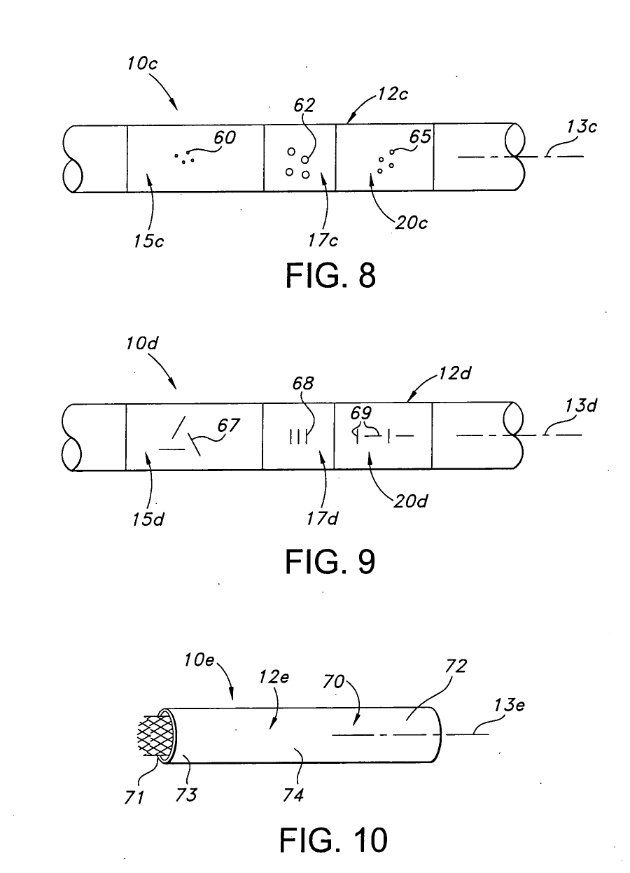

An alternative embodiment of the ePTFE structure 10c is shown in FIG. 8. Parts illustrated in FIG. 8 which correspond to parts illustrated in FIGS. 1 to 3 have, in FIG. 8, the same reference numeral as in FIGS. 1 to 3 with the addition of the suffix “c”. In this alternative embodiment, the first region 15c includes a plurality of resin particles 60 separated from one another by voids. The resin particles 60 each have a first size which is related to the density of the first region 15c. The second region 17c includes a plurality of resin particles 62 separated from one another by voids. The resin particles 62 each have a second size which is related to the density of the second region 17c. The second sizes of the resin particles 62 are greater than the first size of the resin particles 60. Consequently, the density of the second region 17c is greater than the density of the first region 15c because the densities of the respective regions are proportional to the corresponding particle sizes

The third region 20c includes a plurality of resin particles 65 which are separated from one another by voids. The resin particles 65 each have a third size which is related to the density of the third region 20c. The third size of the resin particles 65 is smaller than the second size of the resin particles 62 of the second region 17c. Consequently, the density of the third region 20c is less than the density of the second region 17c because the densities of the respective regions are proportional to the corresponding particle sizes. The third size of the resin particles 65 is larger than the first size of the resin particles 60 of the first region 15c. Consequently, the density of the third region is greater than the density of the first region 15c because the densities of the respective regions are proportional to the corresponding particle sizes. The proportionality between the first, second, and third sizes of the resin particles 60, 62, 65 and the densities of the first, second, and third regions 15c, 17c, 20c provides for an increase in the size of the resin particles to result in an associated increase in the density of the region in which such resin particles are contained.

An alternative embodiment of the ePTFE structure 10d is shown in FIG. 9. Parts illustrated in FIG. 9 which correspond to parts illustrated in FIGS. 1 to 3 have, in FIG. 9, the same reference numeral as in FIGS. 1 to 3 with the addition of the suffix “d”. In this alternative embodiment, the first region 15d includes a plurality of nodes 67 oriented relative to one another by a first orientation. The first orientation of the nodes 67 is triangular as shown in FIG. 9. The first orientation provides corresponding distances between the nodes 67. Smaller and larger distances between the nodes 67 provide increased and reduced densities, respectively, of the first region 15d. The second region 17d includes a plurality of nodes 68 oriented relative to one another by a second orientation. The second orientation of the nodes 68 is transverse as shown in FIG. 9. The second orientation provides corresponding distances between the nodes 68. Smaller and larger distances between the nodes 68 provide increased and reduced densities, respectively, of the second region 17d.

The third region 20d includes a plurality of nodes 69 oriented relative to one another by a third orientation. The third orientation of the nodes 69 alternates between transverse and longitudinal, as shown in FIG. 9. The third orientation provides corresponding distances between the nodes 69. Smaller and larger distances between the nodes 69 provide increased and reduced densities, respectively, of the third region 20d. The orientations which provide the relatively smaller and larger respective distances between the nodes 67, 68, 69 will result in the corresponding regions 15d, 17d, 20d having increased and reduced densities, respectively, relative to the other regions.

The first, second, and third orientations of the nodes 67, 68, 69 may affect the respective INDs which, in turn, affect the density of the first, second and third regions 15d, 17d, 20d. For example, the ePTFE structure 10d may have a non-uniform or random node orientation and have an increased density for a node orientation which is tightly packed. A tightly packed node orientation is provided by a reduced IND. Also, the ePTFE structure 10d may have a uniform microstructure which has a node orientation which is tightly packed resulting in an increased density of the ePTFE structure. Alternatively, the uniform and non-uniform microstructures may have a node orientation which is spread out thinly resulting in a reduced density of the ePTFE structure. A spread out thinly node orientation is provided by an increased IND.

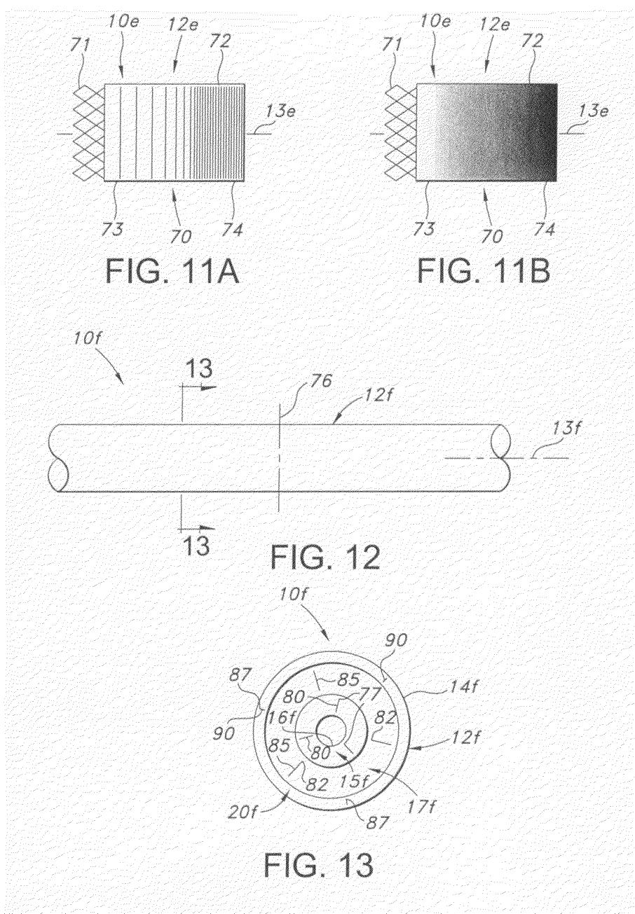

FIGS. 10, 11A, and 11B show a stent-graft composite 70 including an ePTFE tube structure 12e and a stent structure 71 therein. Parts illustrated in FIGS. 10, 11A, and 11B which correspond to parts illustrated in FIGS. 1 to 3 have, in FIGS. 10, 11A, and 11B, the same reference numeral as in FIGS. 1 to 3 with the addition of the suffix “e”. The stent structure 71 is in coaxial relation with the tube structure 12e and fixed thereto.

The stent structure 71 may be formed of materials such as nitinol, elgiloy, stainless steel or cobalt chromium, including NP35N. Additionally, the stent structure 71 may be formed of materials such as stainless steel, platinum, gold, titanium and other biocompatible metals, as well as polymeric stents. Also, the stent structure 71 may be formed of materials including cobalt-based alloy such as Elgiloy, platinum, gold, titanium, tantalum, niobium, and combinations thereof and other biocompatible materials, as well as polymers. Additionally, the stent structure 71 may include structural members which have an inner core formed of tantalum gold, platinum, iridium, or a combination thereof, and an outer cladding of nitinol to provide composite members for improved radio-opacity or visibility. Examples of such composite members are disclosed in U.S. Patent Application Publication 2002/0035396, the entire contents of which are hereby incorporated by reference herein.

The stent structure 71 may have various embodiments. For example, the stent structure 71 may be self-expanding or expandable by a balloon. The stent structure 71 may include one or more coiled stainless steel springs, helically wound coil springs including a heat-sensitive material, or expanding stainless steel stents formed of stainless steel wire in a zig-zag pattern. The stent structure 71 may be capable of radially contracting or expanding, such as by radial or circumferential distension or deformation. Self-expanding stents include stents which mechanically urge the stent to radially expand, and stents which expand at one or more specific temperatures as a result of the memory properties of the stent material for a specific configuration. Nitinol is a material which may be included in the stent structure 71 for providing radial expansion thereof both by mechanical urging, or by the memory properties of the nitinol based on one or more specific temperatures. The stent structure 71 may include one or more of the stents disclosed in U.S. Pat. Nos. 4,503,569, 4,733,665, 4,856,516, 4,580,568, 4,732,152, and 4,886,062, the entire contents of each of which are hereby incorporated by reference herein.

The tubular structure 12e provides a covering for the stent structure 71. The tubular structure 12f has an outer surface 72, the density of which is different depending on the longitudinal position relative to the longitudinal axis 13e. This longitudinal variation in the density of the outer surface 72 may be provided by variations in the microstructure of the ePTFE tubular structure 12e according to the variations of the microstructures of the tubular structures 12, 12a, 12b, 12c, 12d shown in FIGS. 1 to 9.

The tubular structure 12e has an end region 73 which is contiguous with one of the ends thereof. The end region 73 includes a portion of the outer surface 72 which has a relatively low density. The low density of this portion of the outer surface 72 facilitates the ingrowth of tissue which is typically in contact with the outer surface when the stent graft composite 70 is implanted within the body of a patient. The ingrowth of tissue into the outer surface 72 facilitates anchoring of the tubular structure 12e to the tissue. This may also increase the rate of anchoring or securing of the tubular structure 12e to the tissue, which may be beneficial. The enhanced anchoring and securing will typically reduce the likelihood of migration of the stent graft composite 70 through the tissue. The relatively low density of the portion of the outer surface 72 within the end region 73 also typically provides for relatively faster settling and healing characteristics which is beneficial for the patient.

The tubular structure 12e includes an intermediate region 74 which is located longitudinally between the ends thereof. The portion of the outer surface 72 which is included in the intermediate region 74 has a relatively high density which provides enhanced mechanical properties for this portion of the outer surface. These enhanced mechanical properties include strength, and durability.

The density of the outer surface 72 increases continuously from the end region 73 to the intermediate region 74, as illustrated in FIGS. 11A and 11B. The density of the outer surface 72 may correspondingly decrease continuously from the intermediate region 74 to the end region which is contiguous with the other end of the tubular structure 12e. The continuous variation of the density of the outer surface 72 between the end and intermediate regions 73, 74 establishes a longitudinal density gradient through the tubular structure 12e. The density of the tubular structure 12e is substantially uniform between the outer and inner surfaces thereof or transversely relative to the longitudinal axis 13e. In alternative embodiment of the tubular structure 12e, the density thereof may vary between the outer and inner surfaces to establish a radial density gradient through the wall of the tubular structure.

The inner surfaces of the tubular structure 12e and stent structure 71 establish the radial boundary of the lumen and are normally smooth.

In an alternative embodiment of the tubular structure 12e, the portions of the outer surface 72 which have a relatively low density may have longitudinal positions relative to the longitudinal axis 13e which are between the ends of the tubular structure 12e.

An alternative embodiment for the ePTFE structure 10f is shown in FIGS. 12 and 13. Parts illustrated in FIGS. 12 and 13 which correspond to parts illustrated in FIGS. 1 to 3 have, in FIGS. 12 and 13, the same reference numeral as in FIGS. 1 to 3 with the addition of the suffix “f”. In this alternative embodiment, the ePTFE tubular structure 12f has a transverse cross-sectional plane 76. The first region 15f has a first radial position relative to the plane 76 such that the inner surface of the first region is contiguous with the inner surface 16f of the tubular structure 12f. The second region 17f has a second radial position relative to the plane 76 such that the inner surface of the second region is contiguous with the outer surface of the first region 15f. The third region 20f has a third radial position relative to the plane 76 such that the inner surface of the third region is contiguous with the outer surface of the second region 17f. The outer surface of the third region 20f is contiguous with the outer surface 14f of the ePTFE tubular structure 12f.

The first, second, and third regions 15f, 17f, 20f each have corresponding densities which are different from one another. These differences in densities may be provided by differences in the respective node and fibril microstructures of the first, second and third regions 15f, 17f, 20f which correspond to the differences in the node and fibril microstructures of the ePTFE structures 10, 10a, 10b, 10c, 10d. One such difference in the node and fibril microstructure is contained in the ePTFE structure 10 shown in FIG. 3 in which different INDs result in different microstructures, and consequently, different densities. Correspondingly, the differences in the microstructures of the first, second and third regions 15f, 17f, 20f result from the first, second and third regions having corresponding INDs which are different from one another.

The microstructure of the first region 15f includes a plurality of nodes 77 separated from one another by first distance 80. The microstructure of the second region 17f includes a plurality of nodes 82 separated from one another by a second distance 85. The second distance 85 is larger than the first distance 80 which results in the density of the first region 15f being greater than the density of the second region 17f.

The microstructure of the third region 20f includes a plurality of nodes 87 separated from one another by a third distance 90. The third distance 90 is smaller than both the first distance 80 and second distance 85. Consequently, the density of the third region 20f is larger than the densities of the first region 15f and the second region 17f.

The differences in the densities of the first, second and third regions 15f, 17f, 20f may result from differences in characteristics or properties of the respective microstructures other than the INDs, such as the microstructures of the ePTFE structures 10a, 10b, 10c, 10d.

The tubular structure 12f may be assembled to a stent structure located therein and in coaxial relation with the tubular structure to form a stent-graft composite. Such a stent structure may be provided by the stent structure 71. Additionally, portions of the outer surface of the third region 20f may have a relatively low density to facilitate ingrowth, as described for the tube structure 12e. Also, portions of the outer surface of the third region 20f may have a relatively high density for enhanced mechanical properties, as described for the tubular structure 12e.

The ePTFE tubular structures 12, 12a, 12b, 12c, 12d, 12e may be treated with anti-thrombogenic agents (such as heparin, heparin derivatives, urokinase, and PPack (dextrophenylalanine proline arginine chloromethylketone)), anti-proliferative agents (such as enoxaprin, angiopeptin, or monoclonal antibodies capable of blocking smooth muscle cell proliferation, hirudin, and acetylsalicylic acid), anti-inflammatory agents (such as dexamethasone, prednisolone, corticosterone, budesonide, estrogen, sulfasalazine, and mesalamine), antineoplastic/antiproliferative/anti-miotic agents (such as paclitaxel, 5-fluorouracil, cisplatin, vinblastine, vincristine, epothilones, endostatin, angiostatin and thymidine kinase inhibitors), anesthetic agents (such as lidocaine, bupivacaine, and ropivacaine), anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides), vascular cell growth promotors (such as growth factor inhibitors, growth factor receptor antagonists, transcriptional activators, and translational promotors), vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin), cholesterol-lowering agents, vasodilating agents, and agents which interfere with endogenous vascoactive mechanisms.

The tubular structures 12, 12a, 12b, 12c, 12d, 12e are preferably formed of ePTFE. Alternatively, or in combination with ePTFE, the tubular structures 12, 12a, 12b, 12c, 12d, 12e may be formed of biocompatible materials, such as polymers which may include fillers such as metals, carbon fibers, glass fibers or ceramics. Such polymers may include olefin polymers, polyethylene, polypropylene, polyvinyl chloride, polytetrafluoroethylene which is not expanded, fluorinated ethylene propylene copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, copolymers, and combinations thereof. Also, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, and natural silk may be included in the tubular structures 12, 12a, 12b, 12c, 12d, 12e.

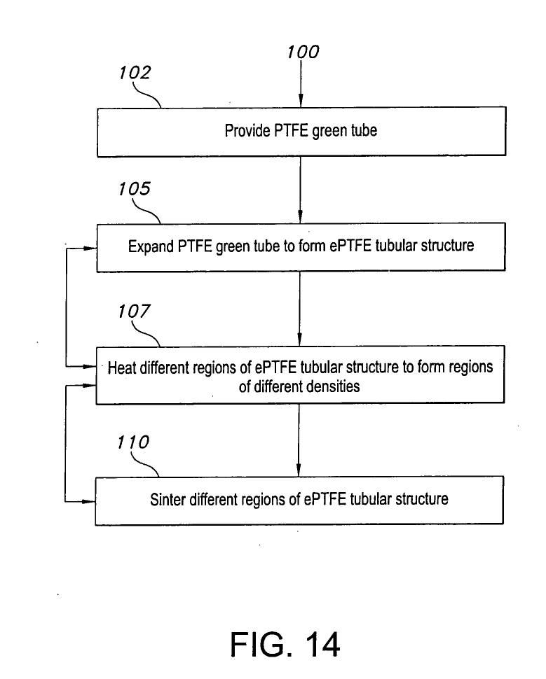

A method 100 for making the ePTFE structure 10, 10a, 10b, 10c, 10d, 10e is represented by the block diagram of FIG. 14. The method 100 includes providing 102 a PTFE structure, such as a PTFE green tube. The method 100 further includes expanding 105 the PTFE structure, such as the PTFE green tube, to form an ePTFE structure, such as an ePTFE tubular structure, which has a node and fibril microstructure. The expansion 105 may be longitudinal or radial or a combination of longitudinal and radial, the latter of which may be referred to as bi-axial.

The method 100 further includes heating 107 different regions of the ePTFE structure, which may be an ePTFE tubular structure, to form regions of different densities therein. The heating 107 may follow the expansion 105 or be concurrent therewith.

The heating 107 of an ePTFE tubular structure may include heating different regions thereof. One embodiment of the heating 107 includes heating 107 a first region of the ePTFE structure which is formed from the expansion 105. The heating 107 provides the first region with a first density, such as the first regions 15, 15a, 15b, 15c, 15d, 15e. The heating 107 may maintain the temperature of the first region, such as the first regions 15, 15a, 15b, 15c, 15d, 15e, at a temperature of about 300 to 600 degrees F. (Fahrenheit) for a duration of about 1 to 90 minutes.

The method 100 further includes heating 107 a second region of the ePTFE structure which is formed from the expansion 105. The heating 107 provides the second region with a second density, such as the second regions 17, 17a, 17b, 17c, 17d, 17e. The heating 107 may maintain the temperature of the second region, such as the second regions 17, 17a, 17b, 17c, 17d, 17e, at a temperature of about 300 to 600 degrees F. for a duration of about 1 to 90 minutes.

The method 100 further includes heating 107 a third region of the ePTFE structure which is formed from the expansion 105. The heating 107 provides the third region with a third density, such as the third regions 20, 20a, 20b, 20c, 20d, 20e. The heating 107 may maintain the temperature of the third region, such as the third regions 20, 20a, 20b, 20c, 20d, 20e, at a temperature of about 300 to 600 degrees F. for a duration of about 1 to 90 minutes.

The method 100 further includes sintering 110 the different regions of the ePTFE structure which are formed during the heating 107. The sintering 110 may follow the heating 107 or be concurrent therewith. The ePTFE structure which is sintered 110 may be an ePTFE tubular structure, such that the sintering 110 includes the sintering of first, second and third regions, such as the first regions 15, 15a, 15b, 15c, 15d, 15e, second regions 17, 17a, 17b, 17c, 17d, 17e, and third regions 20, 20a, 20b, 20c, 20d, 20e.

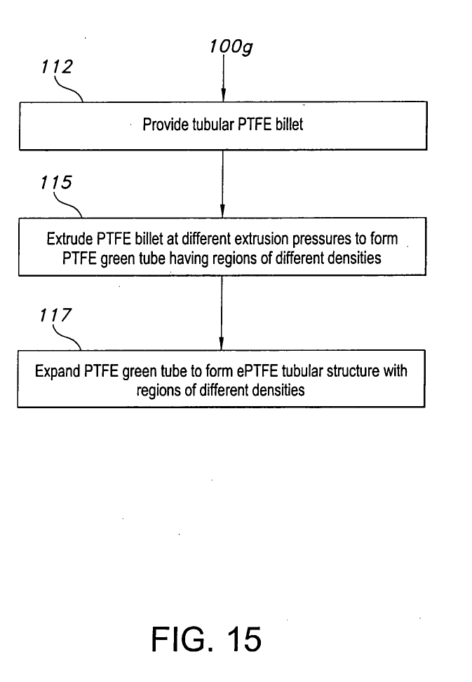

An alternative second embodiment of the method 100g for making the ePTFE structure 10, 10a, 10b, 10c, 10d, 10e is represented by the block diagram of FIG. 15. The method 100g includes providing 112 a PTFE billet. The PTFE billet is extruded 115 to form a PTFE structure having regions each of which has a corresponding density such that the respective densities are different from one another. The PTFE billet which is extruded 115 may be a tubular PTFE billet such that the extrusion forms a PTFE green tube having regions of different densities.

The extrusion 115 results in the formation of a PTFE green tube having regions of different densities. The regions of different densities may be provided by differences in the extrusion pressures which are used to form the respective regions. In one embodiment, the PTFE structure may have a first region which is formed by the application of a first extrusion pressure to the PTFE billet. The PTFE structure may have a second region which is formed by the application of a second extrusion pressure to the PTFE billet. The PTFE structure may have a third region which is formed by the application of a third extrusion pressure to the PTFE billet. The first, second, and third extrusion pressures are different from one another to provide for the densities of the first, second and third regions to be different from one another. One embodiment of the extrusion 115 includes first, second, and third extrusion pressures each being about 100 to 20,000 psi (pounds per square inch).

Following the extrusion 115, the PTFE structure is expanded 117 to form an ePTFE structure which has a node and fibril microstructure. The expansion 117 provides for the ePTFE structure to have first, second and third regions which correspond to the first, second and third regions of the PTFE structure. The expansion 117 further provides for the densities of the first, second and third regions of the ePTFE structure to be different from one another. The expansion 117 may be longitudinal or radial or both radial and longitudinal, the latter of which may be referred to as bi-axial expansion.

The PTFE structure which is expanded 117 may be a PTFE green tube from which the expansion forms an ePTFE tubular structure having regions of different densities. Embodiments of such an ePTFE structure include the ePTFE tubular structures 12, 12a, 12b, 12c, 12d, 12e, which include respective first regions 15, 15a, 15b, 15c, 15d, 15e, second regions 17, 17a, 17b, 17c, 17d, 17e, and third regions 20, 20a, 20b, 20c, 20d, 20e.

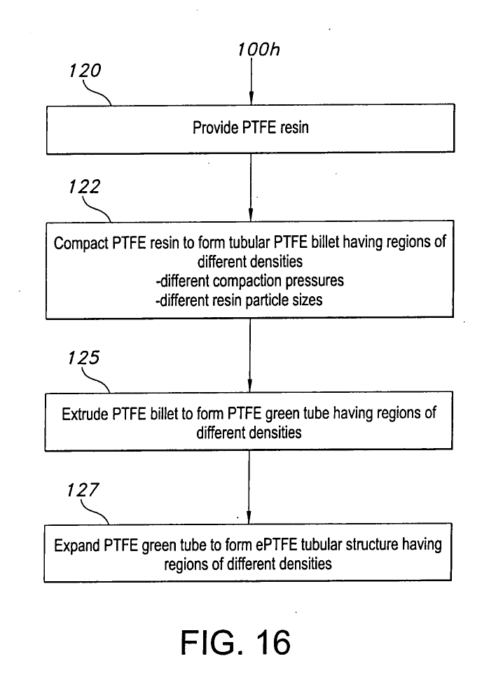

An alternative embodiment for the method 100h is shown in FIG. 16. The method 100h includes providing 120 a PTFE resin. The method 100h further includes compacting 122 the PTFE resin to form a PTFE billet having regions of different densities. The compaction 122 may provide for the PTFE billet to have a tubular structure which has first, second and third regions each of which has a corresponding density such that the densities of the first, second and third regions are different from one another.

The different densities of the respective regions of the PTFE billet may be provided by compacting 122 the PTFE resin at different compaction pressures each of which corresponds to a respective region of the PTFE billet.

The different densities of the respective regions of the PTFE billet which results from the compaction 122 may alternatively be provided by using PTFE resin having different particle sizes each of which corresponds to respective regions of the PTFE billet. For example, the compaction 122 may include compacting particles of the PTFE resin having a first particle size to form the first region. The compaction 122 may further include compacting particles of the PTFE resin having a second particle size to form the second region. The compaction 122 may further include compacting particles of the PTFE resin having a third particle size to form the third region. The first, second and third particle sizes are different from one another which provides for the densities of the first, second, and third regions to be different from one another.

Following the compaction 122, the PTFE billet is extruded 125 to form a PTFE structure having regions of different densities which correspond to the regions of the PTFE billet which have different densities. The extrusion 125 of a PTFE billet including first, second and third regions which have different densities will result in the formation of a PTFE structure having first, second and third regions which correspond to the first, second, and third regions of the PTFE billet. The extrusion 125 provides for the density of the first, second and third regions of the PTFE structure to be different from one another.

The extrusion 125 of a PTFE billet which is tubular results in the formation of a PTFE green tube having regions which correspond to the regions of the PTFE billet which have different densities. The extrusion 125 provides for the densities of the respective regions of the PTFE green tube to be different from one another.

Following the extrusion 125, the PTFE structure is expanded 127 to form an ePTFE structure which has a node and fibril microstructure. The expansion 125 provides for the ePTFE structure to have regions which correspond to the regions of the PTFE structure which have different densities such that the ePTFE structure has respective regions the densities of which are different from one another.

The PTFE structure may be a PTFE green tube the expansion 125 of which results in the formation of an ePTFE tubular structure which has regions of different densities. Embodiments of ePTFE tubular structures which may be made according to the method 100h include the tubular structures 12, 12a, 12b, 12c, 12d, 12e which have respective first regions 15, 15a, 15b, 15c, 15d, 15e, second regions 17, 17a, 17b, 17c, 17d, 17e, and third regions 20, 20a, 20b, 20c, 20d, 20e. The ePTFE structures, including ePTFE tubular structures, made according to the methods 100, 100g, 100h may have any number of regions which have different densities.

The U.S. patent application filed in the U.S. Patent and Trademark Office on even date herewith and entitled “Skewed Nodal-Fibril ePTFE Structure”, having as the inventors Julio Duran, Krzysztof Sowinski, and Jamie S. Henderson, and designated by Attorney Docket No. 760-213 is hereby incorporated by reference herein. The method for forming the skewed nodal-fibril microstructure, including the rotation of a tubular structure, may be used to form ePTFE structures having regions of different densities of the present invention. The regions of different densities may be provided by the regions having different IND's, such as the regions 15d, 17d, 20d, which may be formed by reducing the IND's by varying degrees by rotating or twisting the tubular structure 12d according to the method of the U.S. patent application entitled “Skewed Nodal-Fibril ePTFE Structure” (Attorney Docket No. 760-213). U.S. patent application Ser. No. 11/026,777 filed Dec. 31, 2004, and designated by Attorney Docket No. 760-172 is hereby incorporated by reference herein.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

What is claimed is:

1. An ePTFE structure having a node and fibril micro-structure, said ePTFE structure comprising first and second regions each of which has a corresponding density, said density of said first region being different from said density of said second region.

2. An ePTFE structure according to claim 1, wherein said first region comprises a plurality of nodes separated from one another by a first distance,
said second region comprising a plurality of nodes separated from one another by a second distance,
said second distance being different from said first distance to provide for said density of said first region to be different from said density of said second region.

3. An ePTFE structure according to claim 1, wherein said first region comprises a plurality of nodes each of which has a first size,
said second region comprising a plurality of nodes each of which has a second size,
said second size being different from said first size to provide for said density of said first region to be different from said density of said second region.

4. An ePTFE structure according to claim 1, wherein said first region comprises a plurality of nodes connected to one another by a plurality of fibrils wherein said fibrils each have a first length,
said second region comprising a plurality of nodes connected to one another by a plurality of fibrils wherein said fibrils each have a second length,
said second length being different from said first length to provide for said density of said first region to be different from said density of said second region.

5. An ePTFE structure according to claim 1, wherein said first region comprises a plurality of resin particles separated from one another by voids wherein said resin particles each have a first size,
said second region comprising a plurality of resin particles separated from one another by voids wherein said resin particles each have a second size,
said second size being different from said first size to provide for said density of said first region to be different from said density of said second region.

6. An ePTFE structure according to claim 1, wherein said first region comprises a plurality of nodes oriented relative to one another by a first orientation,
said second region comprising a plurality of nodes oriented relative to one another by a second orientation,
said second orientation being different from said first orientation to provide for said density of said first region to be different from said density of said second region.

7. An ePTFE structure according to claim 1, wherein said ePTFE structure comprises an ePTFE tubular structure,
said ePTFE structure further comprising a stent structure secured to said ePTFE tubular structure,
said ePTFE tubular structure having an inner surface to which said stent structure is secured,
said ePTFE tubular structure having an outer surface at least a portion of which has a relatively low density.

8. An ePTFE structure according to claim 1, wherein said ePTFE structure comprises an ePTFE tubular structure,
said ePTFE tubular structure having a longitudinal axis,
said first and second regions having corresponding first and second longitudinal positions relative to said longitudinal axis,
said first and second longitudinal positions being different from one another.

9. An ePTFE structure according to claim 1, wherein said ePTFE structure comprises an ePTFE tubular structure,
said ePTFE tubular structure having a transverse cross-sectional plane,
said first and second regions having corresponding first and second radial positions relative to said transverse cross-sectional plane,
said first and second radial positions being different from one another.

10. A method for making an ePTFE structure, said method comprising:
providing a PTFE structure;
expanding the PTFE structure to form an ePTFE structure which has a node and fibril micro-structure;
heating a first region of the ePTFE structure such that the first region has a first density; and
heating a second region of the ePTFE structure such that the second region has a second density wherein the second density is different from the first density.

11. A method according to claim 10, and further comprising sintering the ePTFE structure,
said expanding being concurrent with said heating of the first region,
said expanding being further concurrent with said heating of the second region,
said sintering being concurrent with said heating of the first region,
said sintering being further concurrent with said heating of the second region.

12. A method for making a PTFE structure, said method comprising:
providing a PTFE billet;
extruding the PTFE billet to form a PTFE structure having first and second regions each of which has a corresponding density, the density of the first region being different from the density of the second region.

13. A method according to claim 12, wherein said extruding comprises extruding the first region by applying a first extrusion pressure to the PTFE billet,
said extruding further comprising extruding the second region by applying a second extrusion pressure to the PTFE billet,
the second extrusion pressure being different from the first extrusion pressure to provide for the density of the first region to be different from the density of the second region.

14. A method according to claim 12, and further comprising expanding the PTFE structure after said extruding,
said expanding forming an ePTFE structure which has a node and fibril micro-structure having first and second regions which correspond to the first and second regions of the PTFE structure,
said expanding providing for the density of the first region of the ePTFE structure to be different from the density of the second region of the ePTFE structure.

15. A method according to claim 12, wherein said providing comprises providing a PTFE billet having a tubular structure; and
said extruding comprises extruding the PTFE billet to form a PTFE green tube having first and second regions each of which has a corresponding density, the density of the first region being different from the density of the second region.

16. A method for making a PTFE billet, said method comprising:
providing a PTFE resin; and
compacting the PTFE resin to form a PTFE billet having first and second regions each of which has a corresponding density, the density of the first region being different from the density of the second region.

17. A method according to claim 16, wherein said compacting comprises compacting the first region by applying a first compaction pressure to the PTFE resin, and
compacting the second region by applying a second compaction pressure to the PTFE resin,
the second compaction pressure being different from the first compaction pressure to provide for the density of the first region to be different from the density of the second region.

18. A method according to claim 16, wherein said providing comprises providing a PTFE resin including particles which have first and second particle sizes,
said compacting comprises compacting the particles of the PTFE resin which have the first particle size to form the first region, and
compacting the particles of the PTFE resin which have the second particle size to form the second region,
said second particle size being different from first particle size to provide for the density of the first region to be different from the density of the second region.

19. A method according to claim 16, and further comprising extruding the PTFE billet after said compacting,
said extruding forming a PTFE structure having first and second regions which correspond to the first and second regions of the PTFE billet,
said extruding providing for the density of the first region of the PTFE structure to be different from the density of the second region of the PTFE structure.

20. A method according to claim 19, and further comprising expanding the PTFE structure after said extruding,
said expanding forming an ePTFE structure which has a node and fibril micro-structure having first and second regions which correspond to the first and second regions of the PTFE structure,
said expanding providing for the density of the first region of the ePTFE structure to be different from the density of the second region of the ePTFE structure.

21. A method according to claim 16, wherein said compacting comprises compacting the PTFE resin to form a PTFE billet having a tubular structure which includes first and second regions each of which has a corresponding density, the density of the first region being different from the density of the second region.
